(19) United States
(12) Patent Application Publication
(10) Pub. No.: US 2014/0358206 A1
(43) Pub. Date: Dec. 4, 2014

(54) THERAPEUTIC HEATING AND COOLING PACKS COMPRISING DECORATIVE ELEMENTS

(71) Applicant: Laurie Hirokane, Rancho Palos Verdes, CA (US)

(72) Inventors: Laurie HIROKANE, Rancho Palos Verdes, CA (US); David NEEDHAM, Durham, NC (US); Gary FUJII, Rancho Palos Verdes (US)

(73) Assignee: Laurie Hirokane, Rancho Palos Verdes, CA (US)

(21) Appl. No.: 14/293,578

(22) Filed: Jun. 2, 2014

Related U.S. Application Data
(63) Continuation of application No. PCT/US2014/040422, filed on May 31, 2014.
(60) Provisional application No. 61/829,782, filed on May 31, 2013.

(51) Int. Cl. A61F 7/03; A61F 7/10

(52) U.S. Cl. CPC A61F 7/03; A61F 7/106; A61F 2007/0233
USPC 607/109; 607/112

(57) ABSTRACT

It is an object of the invention to provide devices and methods for thermal treatment, e.g., cooling and heating, of body parts for purposes of alleviating discomfort, pain, stress and inflammation.

This application is a continuation of International Application No. PCT/US2014/040422, filed May 31, 2014, which claims priority to U.S. Provisional Patent Application No. 61/829,782, filed May 31, 2013, each of which is hereby incorporated by reference in their entirety.

### BACKGROUND OF THE INVENTION

The following discussion of the background of the invention is merely provided to aid the reader in understanding the invention and is not admitted to describe or constitute prior art to the present invention.

The desirability of using ice bags and heating pads for thermal therapy is well accepted. It has long been an accepted medical practice to apply a cooling element to the surface of a body in the vicinity of an injury to, e.g., reduce swelling. Ice bags enable the user to apply cold to an injury such as a bruise or sprain without unacceptable mess. On the other hand, applying a heating element, such as a “heat pack”, at the appropriate time can also improve the healing process.

A common ice bag that is commercially available is the reusable type comprising a water-impermeable, commonly a rubber-lined, flexible bag having a tubular rigid neck and a removable cap. To use, the bag is filled with ice cubes or ice chips and closed with the cap, then the bag is applied to the bruised area of the body and held it in place by hand. Another type of cold pack that is commercially available is a refrigeratable gel cold pack which comprises a refrigerant or coolant gel material contained in a plastic housing which can be either flexible or relatively inflexible. The gel packs are stored in a freezer for chilling or cooling and are then ready for use. Also commercially available is a chemical pack which comprises two or more pouches for separately storing chemical reactants which can be mixed to produce an endothermic cooling mixture. A common home-use ice bag can be made using a commercially available reclosable plastic sandwich or freezer bag with a zipper seal, such as a Ziploc™ plastic bag filled with ice (Ziploc is a registered trademark of Dow Chemical Company, Midland, Mich.). By “reclosable” it is meant that the bag can be open and closed numerous times.

Similarly, a common hot pack that is commercially available is a gel hot pack which comprises a gel material contained in a flexible plastic housing. The gel packs are heated, e.g., in a microwave oven to a desired temperature and are then ready for use. Also commercially available is a chemical hot pack which comprises two or more pouches for separately storing chemical reactants which can be mixed to produce an exothermic heating mixture. Other hot packs comprise an oxygen activated, heat generating chemical composition contained in a housing that allows air to permeate when heat is needed. A versatile thermal gel pack that is commercially available can serve both as a cooling bag and a heating bag when it is cooled in a household freezer and is heated in a microwave oven, respectively.

One of the disadvantages of many thermal bags is that they need to be inconveniently held by hand to maintain contact with the injured body part. To overcome this inconvenience, several types of thermal bag devices comprising a holder for these bags have been disclosed. Some ice bag devices can be strapped around a body part, with, e.g., loop and hook mating Velcro™-type fastening straps. A cold pack is disclosed having extended perimeter with adhesive means to attach said thermal pack to a skin surface. Yet other means to keep the ice bags in the proximity of the injured body part are also disclosed, such as a jacket with a plurality of attached pockets to hold the thermal packs. These and other ice bag devices are described in detail in the BACKGROUND OF THE INVENTION section of U.S. patent application Ser. Nos. 10/455,885, 10/455,886, and 10/455,888, all filed Jun. 6, 2003. These ice bag devices are usually of complicated design, large and bulky in size and/or expensive to produce and/or difficult to be washed for reuse.

Thermal pack devices that support hot packs or both hot and cold packs are disclosed in U.S. Pat. No. 3,900,035 issued Aug. 19, 1976 to Welch et al.; U.S. Pat. No. 4,527,566 issued Jul. 9, 1985 to Abare; U.S. Pat. No. 4,676,247 issued Jun. 30, 1987 to Van Cleve; U.S. Pat. No. 5,000,176 issued Mar. 19, 1991 to Daniel; U.S. Pat. No. 5,605,144 issued Feb. 25, 1997 to Simmons et al.,; U.S. Pat. No. 5,741,220 issued Apr. 21, 1998 to Brink; and U.S. Pat. No. 6,514,279 B1 issued Feb. 4, 2003 to Lavin, Jr. These thermal pack devices are usually of complicated design, large and bulky in size and/or expensive to produce and/or difficult to be washed for reuse.

### BRIEF DESCRIPTION OF THE INVENTION

It is an object of the invention to provide devices and methods for thermal treatment, e.g., cooling and heating, of body parts for purposes of alleviating discomfort, pain, stress and inflammation.

According to a first aspect of the present invention, there is provided a cooling device adapted to be worn on the body of the user, comprising: at least one reversibly sealable holding element configured for receipt of a replaceable cooling article, the at least one holding element providing a watertight enclosure when sealed; and a cooling article in the form of at least one water-soluble pouch configured to fit within the holding element, the water-soluble pouch comprising one or more compartments containing one or more cooling components sealed within the pouch, wherein the one or more cooling components remain at ambient temperature within the pouch, and when mixed with water, the one or more cooling components and the water provide an endothermic mixture which reaches a temperature of at least about 50° F. (10° C.) for at least about 10 minutes. The cooling device optionally contains at least one ornamental accessory article as defined hereinafter.

According to a second aspect of the present invention, there is provided a heating device adapted to be worn on the body of the user, comprising: at least one reversibly sealable holding element configured for receipt of a replaceable heating article, the at least one holding element providing a watertight enclosure when sealed; and a heating article in the form of at least one water-soluble pouch configured to fit within the holding element, the water-soluble pouch comprising one or more compartments containing one or more heating components sealed within the pouch, wherein the one or more heating components remain at ambient temperature within the pouch, and when mixed with water, the one or more heating components and the water provide an exothermic mixture which reaches a temperature of at least about 40° C. for at least about 10 minutes. The heating device optionally contains at least one ornamental accessory article as defined hereinafter.

The water soluble pouch can comprise one or more discrete compartments. In the case where a plurality of compartments are provided, the compartments may be in a generally superposed or superposable relationship, for example, the plurality of compartments can be symmetrically arranged one above another, side by side (such that they can be folded into a superposed relationship) or any other convenient disposition. Each compartment contains one or more cooling or heating components. Water-soluble pouches comprising a plurality of compartments are herein referred to as multi-compartment pouches.

Multi-compartment pouches are especially advantageous to extend the endothermic or exothermic reaction, as different compartments may be configured to release their contents at different times. By way of example, one compartment may be relatively quickly opened to release its contents when exposed to water, and a second compartment may require additional time of exposure to open and release its contents. One of the two compartments could also comprise separate densified phases allowing delayed or controlled release, for example in the form of micro-beads, noodles or one or more pearlized balls, etc.

In certain embodiments, a pouch has a volume of from about 5 to about 100 mL, preferably from about 15 to about 100 mL, more preferably from about 50 to 100 mL. Beyond 100 mL the device may start to lose its decorative appeal. The dimensions are configured to fit within the holding element providing the watertight enclosure, while still permitting sufficient water to be added to the watertight enclosure so as to support the endothermic or exothermic reaction. In various embodiments, the watertight enclosure is configured to hold a volume of water approximately equal to the pouch volume, about twice the pouch volume, or about three times the pouch volume.

Preferably, the cooling components comprise one or more compounds having a heat of solution of about 15 kJ/mol or greater, i.e., 15 kJ/mol-50 kJ/mol. We calculate that it takes 12 kJ to cool 100 mls water from 30° C.-0° C. Suitable water-soluble pouches include the cooling components in the form of a loose powder, a crystalline form, a densified powder or a tablet. The cooling components (some heats of solution are shown in parentheses along with the moles of material and the mass of material to achieve the 12 kJ of cooling) can comprise one or more compounds selected from the group consisting of C₁₂H₂₂O₁₁, C₆H₁₂O₆, C₆H₁₂O₆.H₂O (19 kJ/mol, 0.63 moles, 125 gms), CO(NH₂)₂ (15 kJ/mol, 0.8 moles, 48 gms), KBr (23 kJ/mol, 0.52 moles, 62 gms), KCl, KClO₃, KMnO₄ (44 kJ/mol, 0.27 moles, 43 gms), KNO₃, NaC₂H₃O₂.3H₂O, NaCl, NaHCO₃, NaNO₃, NH₄Cl, NH₄NO₃ (26 kJ/mol, 0.47 moles, 30 gms), and K₂SO₄. These materials each have a high solubility in water, and the reaction is endothermic, meaning that the material-water system absorbs heat from its surroundings as the material is hydrated. Pouches of the present invention may, for example, comprise one or more compartments, each comprising about 50 to about 200 g urea and about 25 to about 100 g Carbamakool™. In these embodiments, the crystal like urea and Carbamakool™ are mixed together and sealed within a pouch.

The cooling material in the cooling element preferably reaches a temperature of about −5° C. to about 10° C., desirably about −5° C. to about 2° C., within a minute of hydration, and maintains the temperature for a period of about 10 to about 60 minutes, preferably from about 10 to about 30 minutes. Suitable cooling materials are available from Nortech Labs, 125 Sherwood Avenue Farmingdale, N.Y. 11735.

In certain embodiments, the cooling material in the cooling element is used to extend the period in which another chamber remains at temperature. By way of example, the cooling device adapted to be worn on the body of the user may comprise the at least one reversibly sealable holding element configured for receipt of a replaceable cooling article as described above as a first holding element, and a second holding element which contains an ice/water mixture or other material (e.g., a gel) which is previously chilled to a desired temperature (e.g., by placing in a freezer or refrigerator. By introducing cooling components (e.g., comprising one or more compounds having a heat of solution of about 15 kJ/mol or greater) in the first holding element, the endothermic reaction which takes place can act to extend the time at which the material in the second holding element remains cold (e.g., can extend the time at which the ice or gel remains in the frozen state).

Preferably, the heating components comprise one or more compounds having a heat of solution of about −40 kJ/mol to −80 kJ/mol, i.e., they remove heat from their surroundings. We calculate that it takes 8 kJ to heat 100mls of water from 20° C.-40° C. Suitable water-soluble pouches include the heating components in the form of a loose powder, a crystalline form, a densified powder or a tablet. The heating components can comprise one or more compounds selected from the group consisting of CaCl₂ (−82.9 kJ/mol, 0.1 moles, 11 gms), Ca(OH)₂, MgSO₄, HClO₄, KOH, LiBr, LiCl, LiOH, NaOH, NH₃, etc. By way of example, the heating components can be used alone or comprise, in parts by weight, about 40 to about 90 parts anhydrous calcium chloride, about 12 to about 25 parts anhydrous sodium acetate, and about 5 to 15 parts calcium oxide.

The heating material in the heating element preferably reaches a temperature of about 40° C. within a minute of activation, and maintains the temperature for a period of about 10 to about 60 minutes, preferably from about 10 to about 30 minutes.

The pouches may be packed in a string, each pouch being individually separable by a perforation line. Therefore, each pouch can be individually torn-off from the remainder of the string by the end-user. Pouch materials are typically polymeric materials, such as polymers which may be formed into a film or sheet. The pouch material can, for example, be obtained by casting, blow-moulding, extrusion or blown extrusion of the polymeric material, as known in the art.

Preferred polymers, copolymers or derivatives thereof suitable for use as pouch material are selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferred polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and most preferably selected from polyvinyl alcohols, polyvinyl alcohol copolymers and hydroxypropyl methyl cellulose (HPMC), and combinations thereof. Preferably, the level of polymer in the pouch material, for example a PVA polymer, is at least 60%. The polymer can have any average molecular weight, preferably from about 1000 to 1,000,000, more preferably from about 10,000 to 300,000 yet more preferably from about 20,000 to 150,000. Mixtures of polymers can also be used as the pouch material. This can be beneficial to control the mechanical and/or dissolution properties of the compartments or pouch, depending on the application thereof and the required needs. Suitable mixtures include for example mixtures wherein one polymer has a higher water-solubility than another polymer, and/or one polymer has a higher mechanical strength than another polymer. Also suitable are mixtures of polymers having different weight average molecular weights, for example a mixture of PVA or a copolymer thereof of a weight average molecular weight of about 10,000-40,000, preferably around 20,000, and of PVA or copolymer thereof, with a weight average molecular weight of about 100,000 to 300,000, preferably around 150,000.

Also suitable herein are polymer blend compositions, for example comprising hydrolytically degradable and water-soluble polymer blends such as polylactide and polyvinyl alcohol, obtained by mixing polylactide and polyvinyl alcohol, typically comprising about 1-35% by weight polylactide and about 65% to 99% by weight polyvinyl alcohol.

Preferred for use herein are polymers which are from about 60% to about 98% hydrolysed, preferably about 80% to about 90% hydrolysed, to improve the dissolution characteristics of the material.

Most preferred pouch materials are PVA films known under the trade reference Monosol M8630, as sold by Chris-Craft Industrial Products of Gary, Ind., US, and PVA films of corresponding solubility and deformability characteristics. Other films suitable for use herein include films known under the trade reference PT film or the K-series of films supplied by Aicello, or VF-HP film supplied by Kuraray.

In certain embodiments, the holding element may include a lining layer that covers at least the body-facing portion of the cooling or heating element that comes into contact with the patient's skin. The lining layer may be made of fabric, for example a non-woven material, and may generally be used to provide comfort and prevent skin irritation, skin degradation, and potential frostbite or burns. The lining layer may be adhered to the panels or may be detachable. In one embodiment, the lining layer may be comprised of a non-woven material or a film-like material.

In various embodiments, the ornamental accessory article may be in the form of an article of clothing or jewelry which is configured to position the holding element over a body region in need of therapeutic cooling. An article of clothing may be in the form of a shoe, a wrap (such as an elastic bandage or flexible brace), a sleeve which fits over an appendage, a head or neck band, etc. The article of clothing may be provided as a shirt, pants, shorts, etc., comprising a pocket into which the cooling article may be placed. In the form of jewelry, the ornamental accessory may be provided as a necklace, a pendant, etc. This is particularly advantageous when the holding element is positioned to contact the cervical spine.

In other embodiments, the ornamental accessory article may be provided as a decorative object such as a plush animal, a throw pillow, a neck pillow, etc., which is fabricated from a suitable, soft, cloth-like fabric or material for holding against the body.

In related aspects, the present invention relates to a method of manufacturing a therapeutic article, comprising providing a water soluble pouch containing a predetermined quantity of an endothermic or exothermic material, the quantity of material selected to correlate to the volume of a corresponding reversibly sealable holding element providing a watertight enclosure when sealed.

In other related aspects, the present invention relates to a method of manufacturing a therapeutic article, comprising providing a water soluble pouch containing a predetermined quantity of an endothermic or exothermic material; and providing a reversibly sealable holding element providing a watertight enclosure when sealed, the holding element configured to contain a volume of water corresponding to the amount of endothermic or exothermic material contained within the pouch.

In still other related aspects, the present invention relates to a therapeutic method, comprising introducing a water soluble pouch containing a predetermined quantity of an endothermic or exothermic material into a reversibly sealable holding element, wherein the reversibly sealable holding element is a component of a device further comprising at least one ornamental accessory article; and positioning the device on the body of a subject in need of therapeutic heating or cooling.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE FIGURES

FIG. 1 depicts a therapeutic device of the present invention arranged as a collar.

FIG. 2 depicts a therapeutic device of the present invention arranged as a necklace.

FIG. 3 depicts a therapeutic device of the present invention arranged as a neck pillow.

### DETAILED DESCRIPTION

Hot and cold packs are frequently used by athletes and trainers to treat minor injuries, such as, inflammations, sprains, muscle spasms, head-aches, etc. Hot and cold packs consist of two separate compartments, one containing water and the other containing a material. When the material dissolves in water, and this material is for example a salt, the electrostatic attractions between the ions are broken and each ion forms new electrostatic interactions with the water molecules. Whether this process creates an endothermic or exothermic reaction depends on the balance between the ion-ion forces of the solid material that must be overcome and the dipole-ion forces that stabilize the dissolved ions in solution.

When mixed, the two components combine and the material dissolves in the water. This generates a chemical reaction. Depending on the type of material, this reaction can either release or absorb heat energy. When a reaction occurs that releases heat, it is referred to as an exothermic reaction (the prefix exo is Latin for “out of”). In contrast, if the reaction requires heat to occur, it is referred to as an endothermic reaction (the prefix endo is Latin for “into”).

A hot pack is produced if an exothermic reaction occurs as the material and water mix and heat energy is released because this process will raise the temperature of the contents in the pack. A cold pack is produced if an endothermic reaction occurs as the material and water mix and heat energy is absorbed because this process will lower the temperature of the contents in the pack. The amount of heat that is released or absorbed by the packs depends on the concentration of water and materials. As described above, these amounts are related to the amount of heat in kJ required to be added or removed, the number of kJ/mol that the material generates or needs to dissolve, and the molecular weight of the material in order to calculate the weight of material to be included in the pouch for say, 100 mls of water. The table in Appendix 1 shows a few examples (see my spread sheet).

The present invention provides a number of advantages over previous hot and cold packs known in the art. First, the reversibly sealable holding element is reusable upon replacement of the material and water within; second, the pack does not require the use of a freezer or microwave to achieve the desired temperature; third, the water soluble packaging improves handling of the chemicals; fourth, the reversibly sealable holding element and corresponding pouch may be sized to ensure that the correct volumes of material and water are added; fifth, the incorporation of a decorative element makes the devices of the present invention less obtrusive during use.

While the present invention is described in terms of therapeutic devices to apply heat and/or cold to the cervical spine, the skilled artisan will understand that the concepts described herein are generally applicable to other locations on the body.

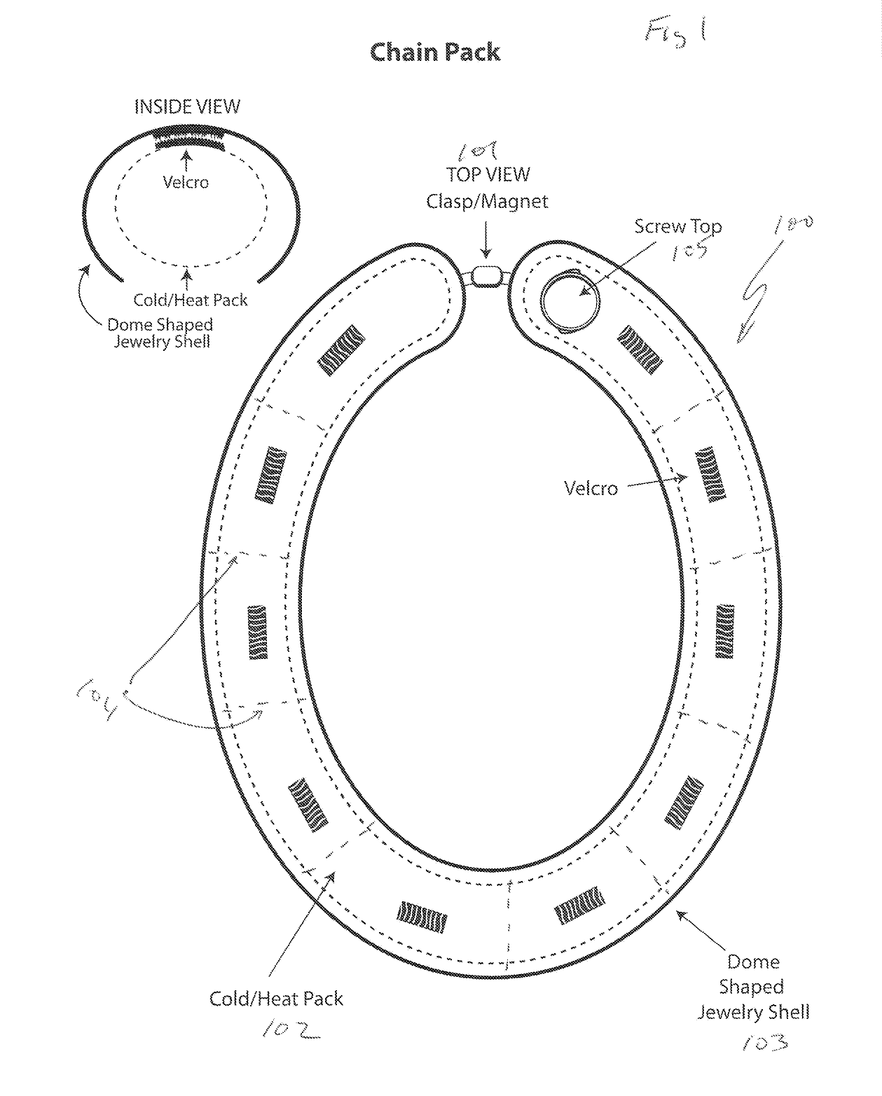

FIG. 1 depicts a therapeutic device 100 of the present invention arranged as a collar. A clasp 101 is used to permit the device to reversibly circle the neck. A chamber 102 is formed within a decorative chamber 103. Chamber 102 is watertight when sealed, and may be reversibly opened in order to introduce one or more water soluble pouches and water, and so that the chamber may be emptied and rinsed following use. The circumference of Chamber 102 may be divided into separate segments by introducing dividers 104. This permits individual locations on the neck to be targeted for therapy. A watertight lid 105 permits access to chamber 102 in order to introduce one or more water soluble pouches and water, and so that the chamber may be emptied and rinsed following use.

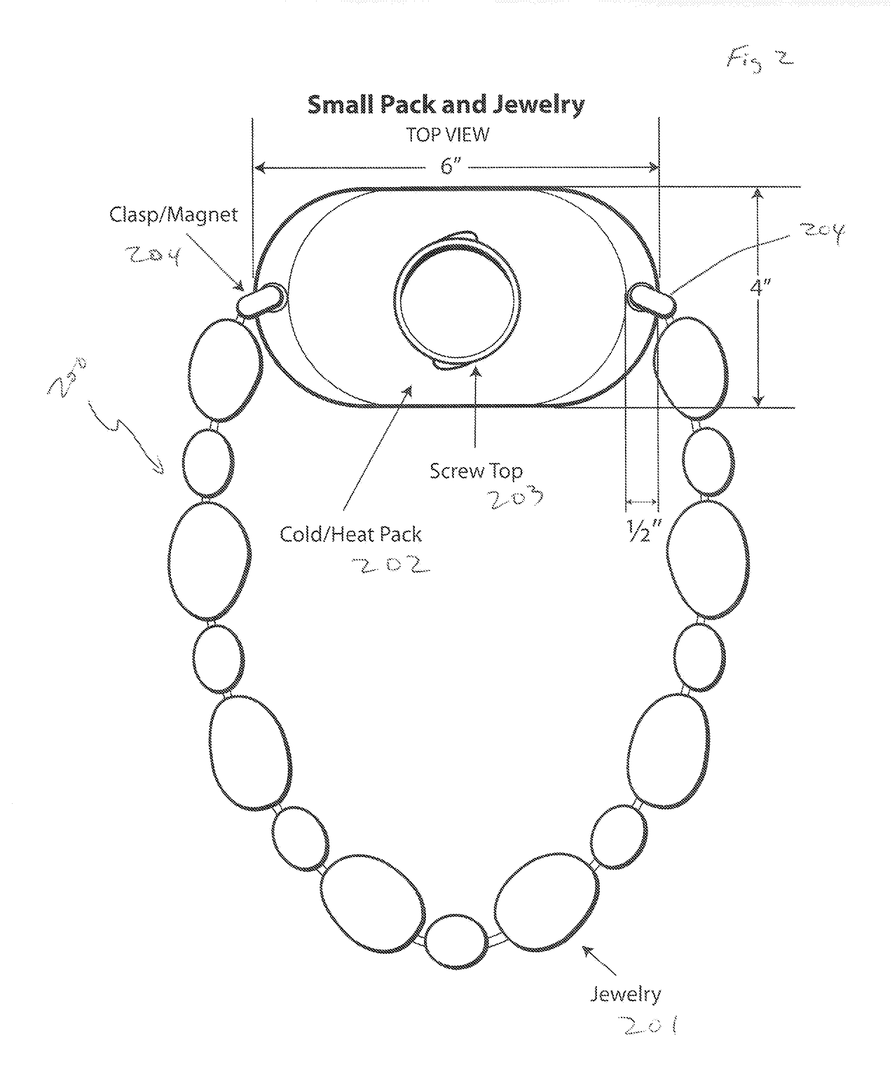

FIG. 2 depicts a therapeutic device in the form of a necklace 200. In this embodiment, a decorative necklace 201 is worn on the front of the body, and the chamber 202 is placed on the back of the neck. A watertight lid 203 permits access to chamber 202 in order to introduce one or more water soluble pouches and water, and so that the chamber may be emptied and rinsed following use. Clasps with magnetic closures 204 permit quick changing of the decorative necklace 201 for alternative appearance.

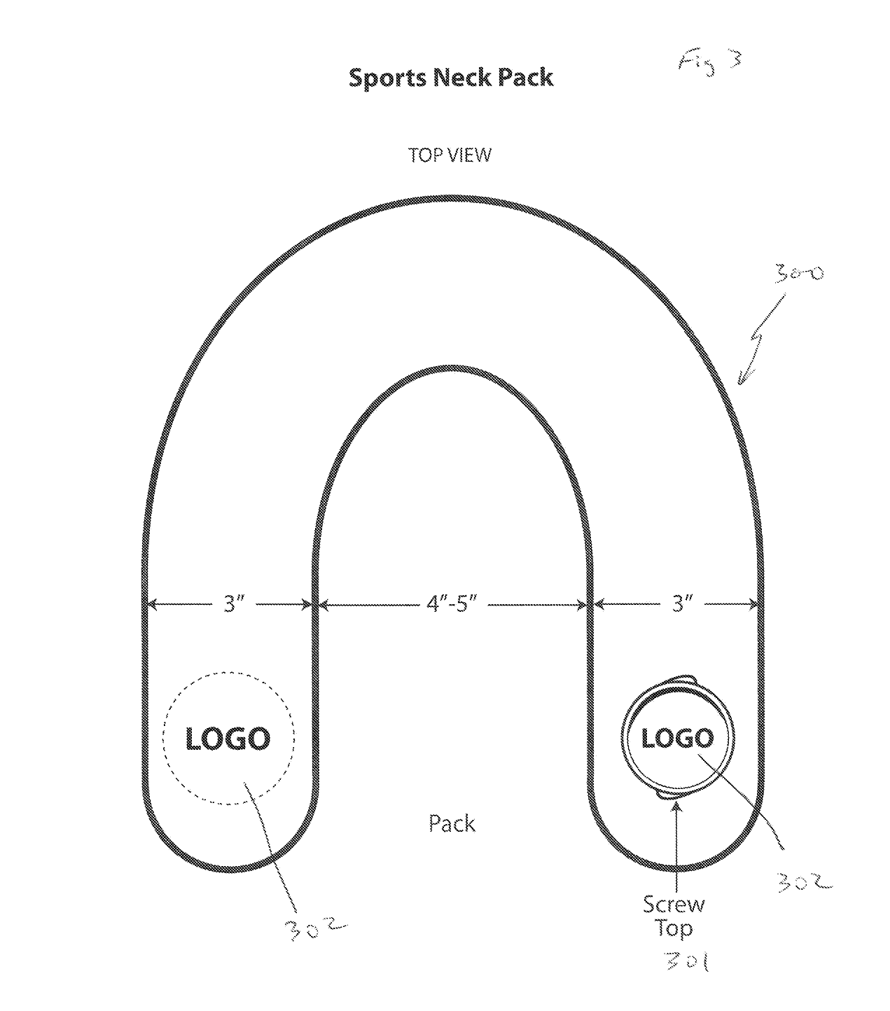

FIG. 3 depicts a therapeutic device in the form of a horseshoe-shaped neck pillow 300. The outer fabric component of the pillow may be adorned with decorative appliqués 302 such as team logos, cartoon characters, etc. A watertight lid 301 permits access to an internal chamber in order to introduce one or more water soluble pouches and water, and so that the chamber may be emptied and rinsed following use.

Suitable materials for use in producing an endothermic (cooling) pouch may be selected from the following table:

| | | |
|---|---|---|
| Material | | |
| solution | Reaction product | Heat of |

| | | | |
|---|---|---|---|
| C₁₂H₂₂O₁₁(s) (sugar) | C₁₂H₂₂O₁₁(aq) | 5.4 | kJ/mol |
| C₆H₁₂O₆(s) (glucose) | C₆H₁₂O₆(aq) | 11 | kJ/mol |
| C₆H₁₂O₆•H₂O(s) | C₆H₁₂O₆•H₂O(aq) | 19 | kJ/mol |
| (glucose monohydrate) | | | |
| CO(NH₂)₂(s) (urea) | CO(NH₂)₂(aq) | 15 | kJ/mol |
| KBr(s) | K⁺(aq) + Br⁻(aq) | 20 | kJ/mol |
| KCl(s) | K⁺(aq) + Cl⁻(aq) | 17 | kJ/mol |
| KClO₃(s) | K⁺(aq) + ClO₃⁻(aq) | 42 | kJ/mol |
| KMnO₄(s) | K⁺(aq) + MnO₄⁻(aq) | 44 | kJ/mol |
| KNO₃(s) | K⁺(aq) + NO₃⁻(aq) | 35 | kJ/mol |
| NaC₂H₃O₂•3H₂O(s) | NaC₂H₃O₂•3H₂O(aq) | 150 | kJ/mol |
| NaCl(s) | Na⁺(aq) + Cl⁻(aq) | 3.9 | kJ/mol |
| NaHCO₃(s) | Na⁺(aq) + HCO₃⁻(aq) | 16.7 | kJ/mol |
| NaNO₃(s) | Na⁺(aq) + NO₃⁻(aq) | 20.4 | kJ/mol |
| NH₄Cl(s) | NH₄⁺(aq) + Cl⁻(aq) | 14.6 | kJ/mol |
| NH₄NO₃(s) | NH₄⁺(aq) + NO₃⁻(aq) | 25.7 | kJ/mol |
| K₂SO₄(s) | 2K⁺(aq) + SO₄²⁻(aq) | 23.8 | kJ/mol |
| K₂SO₄(s) | 2K⁺(aq) + SO₄²⁻(aq) | 23.8 | kJ/mol |
| K₂SO₄(s) | 2K⁺(aq) + SO₄²⁻(aq) | 23.8 | kJ/mol |

Similarly, suitable materials for use in producing an exothermic (heating) pouch may be selected from the following table:

| Material | | | |
|---|---|---|---|
| solution | Reaction product | Heat of | |
| CH₂O₂(l) (methanoic acid) | H⁺(aq) + CHO₂⁻(aq) | −0.86 | kJ/mol |
| C₂H₄O₂(l) (acetic acid) | H⁺(aq) + C₂H₃O₂⁻(aq) | −1.5 | kJ/mol |
| CH₄O(l) (methanol) | CH₄O⁻(aq) | −0.2 | kJ/mol |
| CaCl₂(s) | Ca²⁺(aq) + 2Cl₂⁻(aq) | −82.9 | kJ/mol |
| CaCl₂(s) | CaCl₂ × 2H₂O(aq) | −240 | kJ/mol |
| Ca(OH)₂(s) | Ca²⁺(aq) + 2OH⁻(aq) | −16.2 | kJ/mol |
| CO₂(g) | CO₂(aq) | −19.4 | kJ/mol |
| H₂O₂(l) | H₂O₂(aq) | −3.5 | kJ/mol |
| H₂O₂(l) | H⁺(aq) + OH⁻(aq) | −58 | kJ/mol |
| H₂SO₄(l) | 2H⁺(aq) + SO₄²⁻(aq) | −96.2 | kJ/mol |
| MgSO₄(s) | Mg²⁺(aq) + SO₄²⁻(aq) | −91.2 | kJ/mol |
| HCl(g) | H⁺(aq) + Cl⁻(aq) | −74.8 | kJ/mol |
| HClO₄(l) | H⁺(aq) + ClO₄⁻(aq) | −88.8 | kJ/mol |
| HNO₃(l) | H⁺(aq) + NO₃⁻(aq) | −33.3 | kJ/mol |
| KOH(s) | K⁺(aq) + OH⁻(aq) | −56 | kJ/mol |
| LiBr(s) | Li⁺(aq) + Br⁻(aq) | −49 | kJ/mol |
| LiBr•H₂O(s) | Li⁺(aq) + Br⁻(aq) | −23 | kJ/mol |
| LiBr•2H₂O(s) | Li⁺(aq) + Br⁻(aq) | −9 | kJ/mol |
| LiCl(s) | Li⁺(aq) + Cl⁻(aq) | −37 | kJ/mol |
| LiOH(s) | Li⁺(aq) + OH⁻(aq) | −23.6 | kJ/mol |
| NaOH(s) | Na⁺(aq) + OH⁻(aq) | −44.3 | kJ/mol |
| NH₃(g) | NH₃(aq) | −30.5 | kJ/mol |
| O₂(g) | O₂(aq) | −11.7 | kJ/mol |
| SO₂(g) | SO₂(aq) | −39.5 | kJ/mol |

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

We claim:

1. A cooling device adapted to be worn on the body of the user, comprising:
at least one reversibly sealable holding element configured for receipt of a replaceable cooling article, the at least one holding element providing a watertight enclosure when sealed; and
the cooling article in the form of at least one water-soluble pouch configured to fit within the holding element, the water-soluble pouch comprising one or more compartments containing one or more cooling components sealed within the pouch, wherein the one or more cooling components remain at ambient temperature within the pouch, and wherein when mixed with water, the one or more cooling components and the water provide an endothermic mixture which reaches a temperature of about 10° C. or less for at least about 10 minutes.

2. The cooling device of claim 1, further comprising at least one ornamental accessory article.

3. The cooling device of claim 2, wherein the ornamental accessory article comprises a necklace.

4. The cooling device of claim 3, wherein the ornamental accessory article comprises a pendant.

5. The cooling device of claim 4, wherein the necklace is configured such that when the pendant is worn on the front of the torso, at least one holding element is positioned to contact the cervical spine.

6. The cooling device of claim 1, wherein the ornamental accessory article comprises a neck pillow, and wherein the at least one holding element comprises a reversibly sealable compartment unitary to the neck pillow.

7. The cooling device of claim 1, wherein the cooling components comprise one or more compounds having a heat of solution of about 5 kJ/mol or greater.

8. The cooling device of claim 7, wherein the cooling components comprise one or more compounds selected from the group consisting of C₁₂H₂₂O₁₁, C₆H₁₂O₆, C₆H₁₂O₆.H₂O, CO(NH₂)₂, KBr, KCl, KClO₃, KMnO₄, KNO₃, NaC₂H₃O₂.3H₂O, NaCl, NaHCO₃, NaNO₃, NH₄Cl, NH₄NO₃, and K₂SO₄.

9. The cooling device of claim 1, wherein the water soluble pouch is formed from a material one or more water soluble materials selected from the group consisting of polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, xanthum, carragum, polyacrylates, water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and hydroxypropyl methyl cellulose (HPMC).

10. A heating device adapted to be worn on the body of the user, comprising:
at least one reversibly sealable holding element configured for receipt of a replaceable heating article, the at least one holding element providing a watertight enclosure when sealed; and
the heating article in the form of at least one water-soluble pouch configured to fit within the holding element, the water-soluble pouch comprising one or more compartments containing one or more heating components sealed within the pouch, wherein the one or more heating components remain at ambient temperature within the pouch, and wherein when mixed with water, the one or more heating components and the water provide an exothermic mixture which reaches a temperature of at least about 40° C. for at least about 10 minutes.

11. The cooling device of claim 10, further comprising at least one ornamental accessory article.

12. The heating device of claim 11, wherein the ornamental accessory article comprises a necklace.

13. The heating device of claim 12, wherein the ornamental accessory article comprises a pendant.

14. The heating device of claim 13, wherein the necklace is configured such that when the pendant is worn on the front of the torso, at least one holding element is positioned to contact the cervical spine.

15. The heating device of claim 11, wherein the ornamental accessory article comprises a neck pillow, and wherein the at least one holding element comprises a reversibly sealable compartment unitary to the neck pillow.

16. The heating device of claim 10, wherein the heating components comprise one or more compounds having a heat of solution of about −5 kJ/mol or greater.

17. The heating device of claim 16, wherein the cooling components comprise one or more compounds selected from the group consisting of CaCl₂, Ca(OH)₂, MgSO₄, HClO₄, KOH, LiBr, LiCl, LiOH, NaOH, and NH₃.

18. The heating device of claim 10, wherein the water soluble pouch is formed from a material one or more water soluble materials selected from the group consisting of polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, xanthum, carragum, polyacrylates, water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and hydroxypropyl methyl cellulose (HPMC).

19. A method of locally reducing a temperature at a predetermined location of a subject in need thereof, comprising:
applying a cooling device according claim 1 in a manner configured to reduce the temperature at a local area of the subject.

20. A method of locally increasing a temperature at a predetermined location of a subject in need thereof, comprising:
applying a cooling device according to claim 10 in a manner configured to increase the temperature at a local area of the subject.
